(19) United States
(12) Patent Application Publication
(10) Pub. No.: US 2026/0109784 A1
(43) Pub. Date: Apr. 23, 2026

(54) Composition for Acutely Decreasing Pathophysiologic Effects from a Concussion and a Method of Administering Thereof

(71) Applicant: Marv Enterprises, LLC, Hermitage, PA (US)

(72) Inventor: Mitchell Steven Felder, Hermitage, PA (US)

(21) Appl. No.: 19/115,432

(22) PCT Filed: Sep. 27, 2023
(86) PCT No.: PCT/US2023/033895

§ 371 Date: Mar. 26, 2025

Related U.S. Application Data
(60) Provisional application No. 63/410,405, filed on Sep. 27, 2022.

(51) Int. Cl. C07K 16/44; A61K 9/00; A61K 9/107; A61K 31/568; A61K 31/573; A61K 38/28; A61K 47/44

(52) U.S. Cl. CPC C07K 16/44; A61K 9/0043; A61K 9/107; A61K 31/568; A61K 31/573; A61K 38/28; A61K 47/44

(57) ABSTRACT

Composition for acutely decreasing pathophysiologic effects from a concussion includes a quantity of glutamate monoclonal antibody, a quantity of at least one neurosteroid, a quantity of nasal insulin, a quantity of plant-based oil, and a quantity of saline solution, which are homogenously mixed into a spray composition. This composition may further include a quantity of aspartate monoclonal monoclonal antibody and/or a quantity of monomer. The method of administering the nasal-spray composition is preferably done via a sprayer into each nostril of a concussed patient.

### CROSS-REFERENCE TO RELATED APPLICATIONS

This application is the United States national phase of International Patent Application No. PCT/US2023/033895 filed Sep. 27, 2023, and claims priority to U.S. Provisional Ser. No. 63/410,405 filed Sep. 27, 2022, the disclosures of which are hereby incorporated by reference in their entireties.

### FIELD OF THE INVENTION

The present invention generally relates to a method and a compound for the reduction of the pathophysiologic effects of a concussion, and, more particularly, to a treatment of a concussion using an antibody against glutamate.

### BACKGROUND OF THE INVENTION

Concussions are head injuries in which there may be a loss of consciousness. Concussions cause symptoms such as difficulty with balance, nausea, blurred vision, dizziness, mood dysfunction, sleep disturbances, and memory loss. Common causes of concussions are sports injuries, motor vehicle accidents, bicycle accidents, and falls. Worldwide, concussions are estimated to affect more than 3.5 per 1,000 people a year. Males and young adults are most commonly affected by concussions. A second concussion before the symptoms of a prior concussion have resolved is associated with worse outcomes. Repeated concussions may increase the risk in later life of clinical depression, Parkinson's disease, and chronic traumatic encephalopathy.

### SUMMARY OF THE INVENTION

In summary, one embodiment includes a composition for preventing the pathophysiologic basis of a concussion, including: a targeted antigen antibody against glutamate, a targeted antigen antibody against aspartate, the addition of one or more steroids [i.e., tetrahydrodeoxycorticosterone (THDOC), the androstane 3 alpha-androstanediol, allopregnalone (3 alpha, 5 alpha-THP), pregnenolone (P5)(pregn-5-en-3β-ol-20-one), or combinations thereof], intranasal insulin, a vegetable oil, and a saline solution. The vegetable oil and saline solution are added in order to create a nasal sprayable version of the composition.

Another embodiment provides a method for preventing the pathophysiologic consequences of a concussion including: preparing a composition comprising a monoclonal antibody against glutamate, a targeted antigen antibody against aspartate, intranasal insulin, the addition of one or more neurosteroids [i.e., tetrahydrodeoxycorticosterone (THDOC), the androstane 3 alpha-androstanediol, allopregnalone (3 alpha, 5 alpha-THP), pregnenolone (P5)(pregn-5-en-3β-ol-20-one), or combinations thereof], a vegetable oil, and a saline solution; and spraying the composition in a nasal cavity of a patient.

A further embodiment provides a kit for preventing the pathophysiologic effects of a concussion, including: a composition including, a Glutamate monoclonal antibody capable of restoring glutamate homeostasis in a patient, neurosteroid, nasal insulin, a sesame oil, and a saline solution; and a sprayer for placing the composition in a nasal cavity of the patient.

The foregoing is a summary and thus may contain simplifications, generalizations, and omissions of detail; consequently, those skilled in the art will appreciate that the summary is illustrative only and is not intended to be in any way limiting.

For a better understanding of the embodiments, together with other and further features and advantages thereof, reference is made to the following description, taken in conjunction with the accompanying drawings. The scope of the invention will be pointed out in the appended claims.

### BRIEF DESCRIPTION OF THE DRAWINGS

FIG. 1 is a block diagram illustrating essential constituents of the composition for the present invention.

FIG. 2 is a block diagram illustrating essential and optional constituents of the composition for the present invention.

FIG. 3 is a block diagram illustrating some embodiments for the quantity of at least one neurosteroid.

FIG. 4 is a block diagram illustrating some embodiments for the quantity of plant-based oil.

FIG. 5 illustrates an example flow diagram of a method for reducing the pathophysiologic consequences of a concussion with a nasal spray.

### DESCRIPTION OF THE INVENTION

All illustrations of the drawings are for the purpose of describing selected versions of the present invention and are not intended to limit the scope of the present invention.

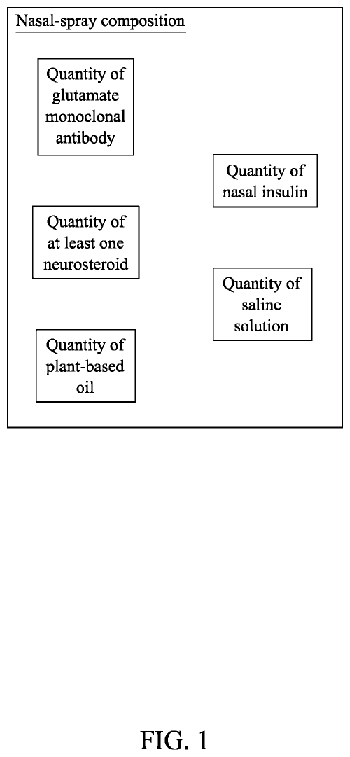

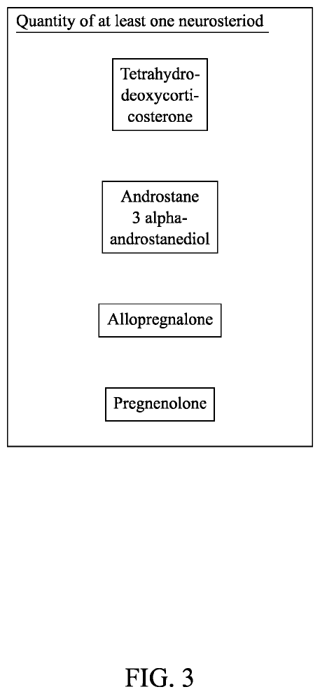

The present invention is a composition for acutely decreasing pathophysiologic effects from a concussion and a method of administering thereof. The composition for the present invention is administered as an aerosol into the nasal cavity of a concussed patient in order to reduce those pathophysiologic effects. As can be seen in FIG. 1, the composition for the present invention comprises a quantity of glutamate monoclonal antibody, a quantity of at least one neurosteroid, a quantity of nasal insulin, a quantity of plant-based oil, and a quantity of saline solution. The quantity of glutamate monoclonal antibody is used to prevent an excessive glutamate physiologic state in a concussed patient. The quantity of glutamate monoclonal antibody is preferably a recombinant monoclonal antibody. The quantity of glutamate monoclonal antibody may specifically react with free L-glutamate. The quantity of glutamate monoclonal antibody may also have minimal cross-reactivity with glutamate residue in a polypeptide chain or proteins. The quantity of at least one neurosteroid allows the composition for the present invention to neuroactively stimulate a concussed patient. The quantity of at least one neurosteroid can be, but is not limited to, tetrahydrodeoxycorticosterone (THDOC), androstane 3 alpha-androstanediol, allopregnalone (3 alpha, 5 alpha-THP), pregnenolone (P5)(pregn-5-en-3β-ol-20-one), or combination thereof, which are shown in FIG. 3. The quantity of nasal insulin is used to reduce the cognitive impairment of a concussed patient. The quantity of plant-based oil and the quantity of saline solution are used as a base to dispense the rest of the composition for the present invention. The quantity of plant-based oil is a hydrophobic element, while the quantity of saline solution preferably has the same osmolarity as bodily tissue, which allows the composition of the present invention to be better absorbed by a concussed patient. Thus, the quantity of glutamate monoclonal antibody, the quantity of at least one neurosteroid, the quantity of nasal insulin, the quantity of plant-based oil, and a quantity of saline solution are homogenously mixed into a nasal-spray composition. The nasal-spray composition is preferably administered as an aerosol via a sprayer into each nostril of the nose for a concussed patient, and 0.5 milliliters (mL) of the nasal-spray composition is released into each nostril. The nasal-spray composition is preferably stored between 1 degrees Centigrade (C) to 10° C.

The aforementioned constituents of the composition for the present invention are also mixed at preferred volumetric proportions. The quantity of glutamate monoclonal antibody is between 0.001 percentage by volume (vol. %) and 0.20 vol. % of the nasal-spray composition at normal temperature and pressure (NTP). The quantity of at least one neurosteroid is between 0.50 vol. % and 10.0 vol. % of the nasal-spray composition at NTP. The quantity of nasal insulin is between 0.10 vol. % and 5.0 vol. % of the nasal-spray composition at NTP. The quantity of plant-based oil is between 10.0 vol. % and 60.0 vol. % of the nasal-spray composition at NTP. The quantity of saline solution is between 15.0 vol. % and 60.0 vol. % of the nasal-spray composition at NTP. In addition, NTP is preferably defined at a temperature of 20° C. and at an absolute pressure of 1 atm.

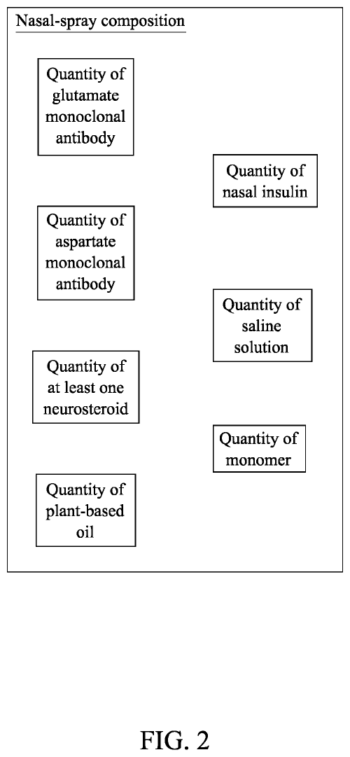

As can be seen in FIG. 2, the composition for the present invention may further comprise a quantity of aspartate monoclonal antibody. The quantity of aspartate monoclonal antibody is used to prevent an excessive aspartate physiologic state in a concussed patient. Similar to the quantity of glutamate monoclonal antibody, the quantity of aspartate monoclonal antibody is preferably a recombinant monoclonal antibody. Moreover, the quantity of aspartate monoclonal antibody is further homogenously mixed into the nasal-spray composition. In terms of a preferred volumetric proportion, the quantity of aspartate monoclonal antibody is between 0.001 vol. % and 0.20 vol. % of the nasal-spray composition at NTP.

As can also be seen in FIG. 2, the composition for the present invention may further comprise a quantity of monomer. The quantity of monomer is used for the slow-release of monoclonal antibodies within a nasal cavity. The quantity of monomer is preferably a biodegradable polymer matrix, which entraps monoclonal antibodies and swell and release the monoclonal antibodies via diffusion within a nasal cavity as the biodegradable polymer matrix is brought into an aqueous environment. Release time can be regulated from hours up to days. The quantity of monomer can be, but is not limited to, DL-lactide, glycolide, ε-Caprolactone, and polyethylene glycol, or combinations thereof. Moreover, the quantity of monomer is further homogenously mixed into the nasal-spray composition. In terms of a preferred volumetric proportion, the quantity of monomer is between 0.50 vol. % and 5.0 vol. % of the nasal-spray composition at NTP.

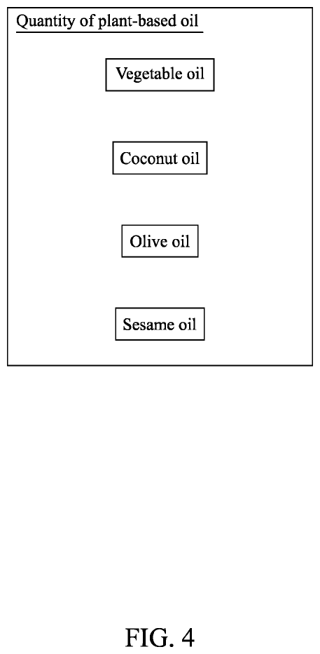

The base of the composition for the present invention can be configured as different embodiments. In some embodiments, the quantity of plant-based oil can be, but is not limited to, vegetable oil, coconut oil, olive oil, sesame oil, or combinations thereof, which is shown in FIG. 4. In some embodiments, the quantity of saline solution is a quantity of sodium chloride and a quantity of isotonic fluid containing water, and the quantity of sodium chloride is preferably 0.9 vol. % of the quantity of saline solution at NTP. In some embodiments, a volumetric ratio at NTP between the quantity of plant-based oil and the quantity of saline solution ranges from 0.1 to 10.

### Supplemental Description

It will be readily understood that the components of the embodiments, as generally described and illustrated in the figures herein, may be arranged and designed in a wide variety of different configurations in addition to the described example embodiments. Thus, the following more detailed description of the example embodiments, as represented in the figures, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, appearances of the phrases “'n one embodiment” or “in an embodiment” or the like in various places throughout this specification are not necessarily all referring to the same embodiment.

Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, etc. In other instances, well-known structures, materials, or operations are not shown or described in detail. The following description is intended only by way of example, and simply illustrates certain example embodiments.

Conventionally, a medical provider diagnosing a concussion may evaluate patient signs and symptoms, review medical history, and conduct a neurological examination. This may be difficult as a concussion may manifest with different severity and symptoms. Also, signs and symptoms of a concussion may not appear until hours or days after the injury. The diagnosis may include a neurological examination, cognitive testing, and/or imaging tests. A neurological examination may include tests for vision, hearing, strength, sensation, balance, coordination, and reflexes. Cognitive testing may include tests for memory, concentration, and the ability to recall information. Brain imaging may be recommended for patients with signs and symptoms such as severe headaches, seizures, repeated vomiting, or symptoms that are becoming worse. Brain imaging may determine whether the injury is severe and has caused bleeding or swelling in the skull. A cranial computerized tomography (CT) scan may be used to assess the brain right after injury using a series of X-rays to obtain cross-sectional images of the skull and brain. Magnetic resonance imaging (MRI) may be used to identify changes in the brain or to diagnose complications that may occur after a concussion. Some patients may be placed under further observation and/or instructed to rest or reduce sensory input. What is needed is a treatment for a concussion that may be acutely administered to reduce pathophysiological effects of the concussion.

Accordingly, described herein is a method and composition for the prevention of the pathophysiologic consequences of a concussion. In an embodiment, the composition may be a nasal spray placed in the nasal cavity of a human. The composition may include a glutamate monoclonal antibody in a fluid. The composition may be sprayed into the nose. The fluid may include at least one vegetable oil and/or a saline solution. In an embodiment, the volumetric ratios of vegetable oil and saline solution in the fluid range from 0.1 to 10. The glutamate antibody may be added to the fluid in a sufficient amount for efficacy to prevent an excessive glutamate physiologic state in the patient. This antibody may be a recombinant monoclonal antibody. The antibody may specifically react with free L-glutamate. The antibody may have minimal cross-reactivity with glutamate residue in the polypeptide chain or proteins.

The illustrated example embodiments will be best understood by reference to the figures. The following description is intended only by way of example, and simply illustrates certain example embodiments.

In an embodiment, a composition comprises a glutamate monoclonal antibody in a fluid. The composition may be sprayed into the nose. The fluid may include at least one vegetable oil and/or a saline solution. In an embodiment, the volumetric ratios of vegetable oil and saline solution in the fluid range from 0.1 to 10.

Glutamate antibody may be added to the fluid in a sufficient amount for efficacy to prevent an excessive glutamate physiologic state in the patient. Preferably, the composition should be refrigerated at a temperature of 1-10° C. prior to spraying into each nostril of the nose. The method may comprise spraying the composition into each nostril and releasing at least about 0.05 mL of the composition into each nostril. This antibody may be a recombinant monoclonal antibody. The antibody may specifically react with free L-glutamate. The antibody may have minimal cross-reactivity with glutamate residue in the polypeptide chain or proteins.

The vegetable oil may include a hydrophobic element. The vegetable oil may be selected from a group consisting of coconut oil, olive oil, sesame oil, and combinations thereof. Most preferably, the vegetable oil consists essentially of sesame oil.

The saline solution comprises a solution of sodium chloride and water. The saline solution may have an osmolarity similar in osmolarity to bodily tissue. The saline solution may comprise a normal saline solution, which is a mixture of salt and water (0.9% NaCl) and is an isotonic fluid containing water, sodium (154 mEq/L) and chloride (154 mEq/L) similar in concentration to bodily fluids.

The glutamate antibody can include a commercially produced monoclonal Glutamate antibody. An alternative glutamate antibody may also be created utilizing standard laboratory procedures well known in the art.

The neurosteroid can include a commercially produced therapeutic. There is the addition of 5-20 mg of one or more neurosteroids such as: tetrahydrodeoxycorticosterone (THDOC), the androstane 3 alpha-androstanediol, allopregnalone (3 alpha, 5 alpha-THP), pregnenolone (P5)(pregn-5-en-3β-ol-20-one), and/or the like.

The nasal insulin can include a commercially produced therapeutic. There is the addition of 20 International units (IU) of nasal insulin.

In embodiments, the composition includes 10-25 ml of sesame oil, at least 5 ml of saline solution, and at least 150 μl of glutamate antibody, 5-20 mg of one or more neurosteroids such as: tetrahydrodeoxycorticosterone (THDOC), the androstane 3 alpha-androstanediol, allopregnalone (3 alpha, 5 alpha-THP),), pregnenolone (P5)(pregn-5-en-3β-ol-20-one), and 20 IU of nasal insulin. In one embodiment, the composition comprises 10 mL of sesame oil and 10 mL normal saline solution. In an alternative embodiment, the composition includes 20 mL of a fluid comprising a 1:1 sesame-oil-to-normal-saline-solution with up to 2000 microliter (μL) of laboratory produced monoclonal glutamate antibody.

In an embodiment, the composition may be created with a longer shelf life. A variant of the composition, for prolongation of its efficacy, utilizes monomers which form a biodegradable polymer matrix for the slow release of the glutamate monoclonal antibodies into the nose. Slow-release drug delivery technologies are well known to one skilled in the art and include commercially available products such as SynBiosys® (InnoCore Pharmaceuticals, Groningen, The Netherlands, a registered trademark in the U.S. and other countries). The technology uses several monomers to form a biodegradable polymer matrix in which monoclonal antibodies against glutamate can be entrapped. When brought into an aqueous environment, the polymers swell and gradually release the monoclonal antibodies by diffusion. Release time can be regulated from hours up to days. The SynBiosys® polymers may comprise DL-lactide, glycolide, Ecaprolactone, and polyethylene glycol (i.e., biological monomers). A SynBiosys®′ feature is that it allows sustained release of the composition over time.

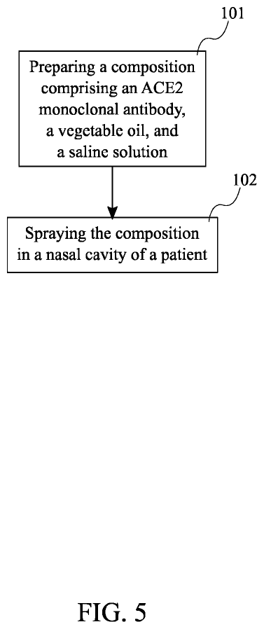

Referring to FIG. 5, an example method is illustrated. In an embodiment, a method for acutely ameliorating the pathophysiologic effects of a concussion may include preparing a composition comprising a glutamate monoclonal antibody, one or more neurosteroid(s), nasal insulin, a vegetable oil, and a saline solution at 101. In an embodiment, the composition may be sprayed in a nasal cavity of a patient at 102.

An exemplary embodiment has been described above. However, it will be apparent to those skilled in the art that numerous variations of the type described could be made to the present invention without departing from the spirit of the invention. The scope of the present invention is defined by the broad general meaning of the terms in which the claims are expressed.

The various embodiments described herein thus represent a technical improvement to concussion treatment. Using the techniques as described herein, an embodiment may use an antibody or antibody compound to treat a concussion in a patient. This is in contrast to conventional methods with limitations mentioned above. Such techniques provide a compound and method for the concussion treatment.

It is noted that the values provided herein are to be construed to include equivalent values as indicated by use of the term “about”. The equivalent values will be evident to those having ordinary skill in the art, but at the least include values obtained by ordinary rounding of the last significant digit.

This disclosure has been presented for the purposes of illustration and description but is not intended to be exhaustive or limiting. Many modifications and variations will be apparent to those of ordinary skill in the prior art. The example embodiments were chosen and described in order to explain principles and practical application, and to enable others of ordinary skill in the art to understand the disclosure for various embodiments with various modifications as are suited to the particular use contemplated.

Thus, although illustrative example embodiments have been described herein with reference to the accompanying figures, it is to be understood that this description is not limiting and that various other changes and modifications may be affected therein by one skilled in the art without departing from the scope or spirit of the disclosure.

Although the invention has been explained in relation to its preferred embodiment, it is to be understood that many other possible modifications and variations can be made without departing from the spirit and scope of the invention as hereinafter claimed.

1-20. (canceled)

21. A composition for acutely decreasing pathophysiologic effects from a concussion comprising:
a quantity of glutamate monoclonal antibody;
a quantity of at least one neurosteroid;
a quantity of nasal insulin;
a quantity of plant-based oil;
a quantity of saline solution;
the quantity of glutamate monoclonal antibody, the quantity of at least one neurosteroid, the quantity of nasal insulin, the quantity of plant-based oil, and a quantity of saline solution being homogenously mixed into a nasal-spray composition; and
the quantity of glutamate monoclonal antibody being between 0.001 percentage by volume (vol. %) and 0.20 vol. % of the nasal-spray composition at normal temperature and pressure (NTP).

22. The composition for acutely decreasing pathophysiologic effects from a concussion as claimed in claim 21, wherein the quantity of at least one neurosteroid is between 0.50 vol. % and 10.0 vol. % of the nasal-spray composition at NTP.

23. The composition for acutely decreasing pathophysiologic effects from a concussion as claimed in claim 21, wherein the quantity of nasal insulin is between 0.10 vol. % and 5.0 vol. % of the nasal-spray composition at NTP.

24. The composition for acutely decreasing pathophysiologic effects from a concussion as claimed in claim 21, wherein the quantity of plant-based oil is between 10.0 vol. % and 60.0 vol. % of the nasal-spray composition at NTP.

25. The composition for acutely decreasing pathophysiologic effects from a concussion as claimed in claim 21, wherein the quantity of saline solution is between 15.0 vol. % and 60.0 vol. % of the nasal-spray composition at NTP.

26. The composition for acutely decreasing pathophysiologic effects from a concussion as claimed in claim 21, further comprising:
a quantity of aspartate monoclonal antibody; and
the quantity of aspartate monoclonal antibody being further homogenously mixed into the nasal-spray composition.

27. The composition for acutely decreasing pathophysiologic effects from a concussion as claimed in claim 26, wherein the quantity of aspartate monoclonal antibody is between 0.001 vol. % and 0.20 vol. % of the nasal-spray composition at NTP.

28. The composition for acutely decreasing pathophysiologic effects from a concussion as claimed in claim 26, wherein the quantity of aspartate monoclonal antibody is a recombinant monoclonal antibody.

29. The composition for acutely decreasing pathophysiologic effects from a concussion as claimed in claim 21, further comprising:
a quantity of monomer; and
the quantity of monomer being further homogenously mixed into the nasal-spray composition.

30. The composition for acutely decreasing pathophysiologic effects from a concussion as claimed in claim 29, wherein the quantity of monomer is between 0.50 vol. % and 5.0 vol. % of the nasal-spray composition at NTP.

31. The composition for acutely decreasing pathophysiologic effects from a concussion as claimed in claim 29, wherein the quantity of monomer is a biodegradable polymer matrix.

32. The composition for acutely decreasing pathophysiologic effects from a concussion as claimed in claim 29, wherein the quantity of monomer is selected from a group consisting of: DL-lactide, glycolide, ε-Caprolactone, and polyethylene glycol, and combinations thereof.

33. The composition for acutely decreasing pathophysiologic effects from a concussion as claimed in claim 21, wherein the glutamate monoclonal antibody is a recombinant monoclonal antibody.

34. The composition for acutely decreasing pathophysiologic effects from a concussion as claimed in claim 21, wherein the quantity of at least one neurosteroid is selected from a group consisting of: tetrahydrodeoxycorticosterone, androstane 3 alpha-androstanediol, allopregnalone, pregnenolone, and combination thereof.

35. The composition for acutely decreasing pathophysiologic effects from a concussion as claimed in claim 21, wherein the quantity of plant-based oil is selected from a group consisting of: vegetable oil, coconut oil, olive oil, sesame oil, and combinations thereof.

36. The composition for acutely decreasing pathophysiologic effects from a concussion as claimed in claim 21, wherein a volumetric ratio at NTP between the quantity of plant-based oil and the quantity of saline solution ranges from 0.1 to 10.

37. The composition for acutely decreasing pathophysiologic effects from a concussion as claimed in claim 21, wherein the quantity of saline solution is a quantity of sodium chloride and a quantity of isotonic fluid containing water.

38. The composition for acutely decreasing pathophysiologic effects from a concussion as claimed in claim 37, wherein the quantity of sodium chloride is 0.9 vol. % of the quantity of saline solution at NTP.

39. The composition for acutely decreasing pathophysiologic effects from a concussion as claimed in claim 21, wherein an osmolarity of the quantity of saline solution is equal to an osmolarity of bodily tissue.
